(19) United States
(12) Patent Application Publication
(10) Pub. No.: US 2014/0276688 A1
(43) Pub. Date: Sep. 18, 2014

(54) System And Method For Simultaneous Laser Cutting And Cauterization Of Tissue

(71) Applicant: Lawrence Livermore National Security, LLC, Livermore, CA (US)

(72) Inventor: Stavros DEMOS, Livermore, CA (US)

(21) Appl. No.: 14/200,169

(22) Filed: Mar. 7, 2014

Related U.S. Application Data
(60) Provisional application No. 61/798,154, filed on Mar. 15, 2013.

(51) Int. Cl. A61B 18/20

(52) U.S. Cl. CPC A61B 18/20
USPC 606/11

(57) ABSTRACT

A method is disclosed for substantially cutting and cauterizing tissue during a surgical procedure. The method involves using a first laser beam to irradiate a first area. The first laser beam is configured in intensity and duration to cut the tissue within the first area, and further causes a temporary increase in an absorptivity of tissue in a second area in proximity to the first area. A second laser beam is used to irradiate the second area while the temporary increase in absorptivity is occurring. The second laser beam has a different intensity than the first laser beam and cauterizes tissue within the second area substantially simultaneously with the cutting of the tissue in the first area.

### CROSS-REFERENCE TO RELATED APPLICATIONS

This application claims the benefit of U.S. Provisional Application No. 61/798,154, filed on Mar. 15, 2013. The entire disclosure of the above application is incorporated herein by reference.

### STATEMENT OF GOVERNMENT RIGHTS

The United States Government has rights in this invention pursuant to Contract No. DE-AC52-07NA27344, between the U.S. Department of Energy and Lawrence Livermore National Security, LLC, for the operation of Lawrence Livermore National Laboratory.

### FIELD

The present disclosure relates to laser systems, and more particularly to a laser system that enables virtually simultaneous cutting and cauterization of tissue.

### BACKGROUND

The statements in this section merely provide background information related to the present disclosure and may not constitute prior art.

Laser surgery is a well-established technique that enables cutting of soft or hard tissue. Usage of lasers has become ubiquitous in wide ranging surgical and microsurgical applications. Laser surgery involves using a laser pulse (or pulses) to cut soft or hard tissue. In simple terms, the pulse laser beam deposits locally, to a well-defined area of tissue, sufficient energy to vaporize small sections of tissue with each pulse. Currently laser surgery is commonly used on the eye to correct near and far-sightedness in vision, and permanently reshapes the cornea. Lasers are also used for the treatment/reduction of enlarged prostates. The use of lasers in dentistry is also expanding.

The potential of laser surgery to expand into various other medical applications is tremendous but there are a number of limitations that need to be resolved. Some of these limitations are technical, while still others relate to the cost and training of using a laser for a medical or dental application. However, a significant limitation of present day laser systems used in medical applications is the inability of the laser to simultaneously cut and cauterize tissue. Cauterization is associated with the “burning” (raising temperature of tissue above a certain threshold) of part of a tissue structure in an attempt to mitigate damage to surrounding tissues, to remove an undesired growth or tumor, or to minimize other potential medical harmful possibilities such as infections or bleeding.

When performing surgical procedures, cauterization of tissue during surgery is associated with depositing heat to the tissue to stop bleeding or for other therapeutic purposes. The main forms of cauterization used today are electrocautery and chemical cautery. Cauterization requires an increase of the tissue temperature to induce permanent alterations with clinically desirable properties. Cauterization must also be “localized,” meaning it must be applied to a small region of tissue around the tissue being cut. Cauterization is typically performed by placing a heated metal object in contact with the tissue to be cauterized to deposit energy into a localized region of tissue via heat conduction. Using a laser to achieve such a process would require a very short penetration depth and relatively low energy deposition rate from the laser to the tissue over relatively long periods of time. However, cutting tissue with a laser is typically associated with depositing a pulse of laser energy to a location which is sufficiently high to cause what may be termed a “micro-explosion.” The micro-explosion tears apart, over small spatial scales, a highly localized region of the tissue where the laser energy is being deposited. But the laser-induced micro-explosion, while serving to cut tissue, is not able to deposit sufficient energy to achieve cauterization of the tissue in surrounding areas of tissue which are in close proximity to the tissue being cut.

The cauterization process, from a technical standpoint, is the complete opposite of the laser cutting process. In the latter case the tissue is quickly brought above the boiling temperature so that there is an explosive reaction (i.e., evaporation, fragmentation and tearing) of the tissue. This can be done over small-localized regions by focusing the laser beam as needed. For example, if using a pulsed laser, the energy coupling from the laser into the tissue leads to a cascade increase of the localized tissue absorptivity, which leads to formation of plasma and localized temperatures on the order of 1 eV or higher, depending on the laser pulse length. This energy leads to the launching of a shockwave (i.e., pressure wave) and heating of the surrounding tissue via heat diffusion. However this heating, although extremely high at the cutting (laser focal) point, is very quickly diminished via heat diffusion. Thus it is not able to cause cauterization over a sufficiently deep surrounding tissue layer.

The above discussion highlights two issues. First, that laser cutting and laser cauterization are completely different processes and that they are applied over different time scales and require completely different laser energy deposition mechanisms. In simple terms, the cutting of tissue requires a short laser pulse of high peak intensity while the cauterization of tissue requires a long laser pulse of low peak intensity. The technology of the laser tissue “cutting” is well developed. The technology of laser heating of a tissue is also in the research/developmental stage of research and development and is often found under the “laser welding” developmental effort. The later process is slow as it requires temperature controlled heating of large areas. Increase of the laser energy to increase the speed leads to adverse effects as the laser energy expands and heats larger than desired areas to higher temperatures. An effective cauterization process needs to be able to provide highly localized, “slow” heating at the area of tissue exposed to the laser cutting micro-explosion.

Accordingly, with presently available technology, typically a separate instrument is required to perform the cauterization of tissue. As one can appreciate, this complicates the performance of any medical procedure, and especially a surgical procedure.

### SUMMARY

In one aspect the present disclosure relates to a method for substantially cutting and cauterizing tissue during a surgical procedure. The method may comprise using a first laser beam to irradiate a first area, the first laser beam being configured in intensity and duration to cut the tissue within the first area, the first laser beam further causing a temporary increase in an absorptivity of tissue in a second area which is in proximity to the first area. A second laser beam is used to irradiate the second area while the temporary increase in absorptivity is occurring. The second laser beam has a different intensity than the first laser beam to cauterize tissue within the second area substantially simultaneously with the cutting of the tissue in the first area.

In another aspect the present disclosure relates to a method for substantially cutting and cauterizing tissue during a surgical procedure. The method may comprise using a first laser beam having a first series of laser pulses to irradiate a first area. The first laser pulses may be configured in intensity and duration to cut the tissue within the first area. The first laser beam may further cause a temporary increase in an absorptivity of tissue in a second area which is in proximity to the first area. A second laser beam may be used which comprises a second series of laser pulses to irradiate the second area while the temporary increase in absorptivity is occurring. The second series of laser pulses is made up of pulses that each have a lower intensity than the pulses of the first series laser pulses, and each may be longer in duration than those of the first series of laser pulses. The second series of laser pulses cauterizes tissue within the second area substantially simultaneously with the cutting of the tissue in the first area.

In still another aspect the present disclosure relates to a system for substantially cutting and cauterizing tissue during a surgical procedure. The system may comprise at least one laser configured to generate a first laser beam and a second laser beam. The first laser beam may be configured in intensity and duration to cut tissue within a first area. The first laser beam further causes a temporary increase in an absorptivity of tissue in a second area which is in proximity to the first area. The second laser beam may be configured in intensity and duration to irradiate the second area while the temporary increase in absorptivity is occurring, the second laser beam having laser pulses which are of a different intensity and a different duration than laser pulses forming the first laser beam, to cauterize tissue within the second area substantially simultaneously with the cutting of the tissue in the first area.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a high level block diagram of a system laser system for both cutting and cauterizing tissue which makes use of two computer controller lasers, or optionally one computer controller laser;

FIG. 2 is a high level block diagram of a system for both cutting and cauterizing tissue which makes use of a single lasing system to generate two distinct types of laser pulses that are used for simultaneous, or virtual simultaneous, cutting and cauterizing of tissue;

FIG. 3 is a high level flowchart setting forth various operations that may be performed using either of the systems of FIG. 1 or 2 to perform both laser cutting and laser cauterizing of tissue; and

FIG. 4 is a graph illustrating one laser pulse shape that may be emitted by a single lasing device or the sum of the emitted light by separate laser devises to produce the two distinct intensity/duration profiles needed for both cutting and cauterizing.

### DETAILED DESCRIPTION

The various embodiments and methodologies of the present disclosure teach rapid cutting and localized cauterization of the tissue immediately after cutting. The system and method takes advantage of distinct processes associated with the material (i.e., tissue) response to the localized heating that can be utilized and integrated into a single instrument that offers the aforementioned capability.

The process of tissue cutting with a laser utilizes a single laser pulse or a train of laser pulses to deliver energy at localized regions, thus increasing the localized temperature on the order of 1 eV (10000 K) or higher, depending on the laser pulse characteristics. The energy deposition is followed by a micro explosion, material evaporation and/or ejection and localized heating of the surrounding region. A key to this invention is the recently improved understanding of transient material changes after exposure to laser-induced high temperatures and pressures. More particularly, it has been discovered that materials (including tissues) experience transient changes relative to their optical properties. More specifically, the affected tissue exhibits increased absorption over periods of time that can extend to 10 to 100 microseconds or longer. This relatively short time of increased absorptivity is localized in the tissue volume around the site of micro-explosion, thus in the region that must be cauterized after laser cutting. For ease of explanation, this modified, localized region of the tissue that exhibits the increased absorptivity due to its exposure, via secondary mechanisms (not from direct exposure), to the laser cutting pulse energy may be termed the “surrounding modified tissue.” The recognition of this transient modification of the absorptive property of the surrounding modified tissue is highly important to the present disclosure. This is because the recognition of particular absorptive property of the surrounding modified tissue, during the relatively small time “window” that laser cutting of the tissue is occurring, enables cauterization of the surrounding modified tissue if a secondary delivery of laser energy is applied to the surrounding modified tissue using a lower intensity laser beam. Accordingly, cauterization of the surrounding modified tissue may be effected simultaneously, or virtually simultaneously, by using a laser beam of a second (i.e., lower) intensity pulse immediately following culling of the tissue using a first (i.e., higher intensity) laser pulse.

Referring to FIG. 1 a system 10 is illustrated for performing both laser cutting and cauterization simultaneously or virtually simultaneously. The system 10 may include a lasing system 12 that is controlled by a suitable computer 14 or other type of suitable electronics. The lasing system 12 in this embodiment makes use of two lasers 12a and 12b. Laser 12a is used to generate a first laser beam comprising laser pulses 16 having a first intensity and a first duration. Laser 12b is used to generate a second laser beam having laser pulses 18 with a second intensity and a second duration. The laser pulses 16 may be in the duration of typically less than 1 microsecond but more optimally on the order of 10 picoseconds to 10 nanoseconds. The laser pulses 18 may be for durations of between about 10-100 microseconds or longer, but otherwise generally in accordance with the duration of the enhanced absorptivity of the surrounding modified tissue. The two pulses 16 and 18 can have the same operational wavelength or they may have different operational wavelengths. In either event the two pulses 16 and 18 will be focused on the same approximate region of tissue. While only a single one of each of the pulses 16 and 18 is shown in FIG. 1, it will be appreciated that in practice a series of pulses 16 followed by a series of pulses 18 may be applied by the lasers 12a and 12b. The pulses 16 and 18 may be applied alternately or they may overlap at times.

It is also important to note that the enhanced absorptivity of the surrounding modified tissue is well above the level of a “background” absorptivity of the “unmodified” tissue. The unmodified tissue will be understood as that tissue which is not being irradiated or otherwise affected/influenced by the first laser pulses 16.

The energy density deposited by the laser pulse 16 in the tissue depends on a multiplicity of parameters but in general it is proportional to the laser intensity and material density. The energy deposited by the laser pulse 18 of the second laser beam is proportional to the laser intensity and the transient tissue absorptivity. However, the intensity of the laser pulse 18 is kept well below the intensity level that could significantly increase the temperature of the surrounding unaffected normal tissue but, due to the increased absorptivity of the surrounding modified tissue, the intensity will still be sufficient to deposit a much higher energy density into the surrounding modified tissue. The laser pulse 18 of the second laser beam may be generated (initiated) before or during each pulse 16 of the first laser beam. The pulse 18 may also be generated (initiated) after the first pulse 16 occurs, but the pulse 18 still needs to be generated within the relatively narrow time window when the enhanced absorption of the surrounding modified tissue is occurring.

FIG. 2 shows a system 100 in accordance with another embodiment of the present disclosure in which a lasing system 102 is controlled by a suitable computer 104. The lasing system 102, however, may generate a laser beam having pulses 106 that each have a shape that accomplishes both the required high intensity, short duration heating for a cutting action on tissue, as well as the lower intensity, longer duration heating required for cauterization. A single one of the pulses 106 is shown in greater detail in FIG. 4. In FIG. 4 pulse 106 has a first portion 106a which forms a spike having a high intensity and short duration, and a second portion 106b that forms a “tail.” The second portion (i.e., tail) 106b is of significantly lower intensity and longer duration than the first portion. The pulse 106 may be applied repeatedly at a desired frequency as needed to accomplish both cutting and cauterization of tissue. As with the system 10, the pulses 106 need to be applied within the narrow time window when the enhanced absorptivity is occurring in the surrounding modified tissue. This time window may be represented as the period 106c of the pulse 106. The pulse 106 may also represent the sum of laser energy emitted by pulses 16 and 18 of the two lasers 12a and 12b of FIG. 1.

It will be appreciated that a method performed using either of the systems 10 or 100 will deliver increased laser energy on the tissue region that requires cauterization (i.e., the area/region in close proximity to the area being cut) while it will minimally affect other tissue areas. This provides a number of important benefits. For one, it enables energy to be deposited in the target tissue region that requires cauterization while the background tissue is not exposed to any potentially harmful level of laser radiation. It also enables the rate of energy deposition to be controlled by the laser intensity, which in turn allows one to control the cauterization process. Still further, the energy deposition by the second laser beam will delay the relaxation of the enhanced, transient absorption that is occurring in the surrounding modified tissue, which in turn can be used to achieve the desired level of cauterization.

Referring to FIG. 3 a flowchart is presented showing a method 200 which may be used to perform simultaneous, or virtually simultaneous, cutting and cauterization of tissue. At operation 202 a first laser beam is generated having a pulse of a first intensity and a first duration, and then applied to an area of tissue to be cut. At operation 204 either prior to, concurrently with, or subsequent to application of the pulse using the first laser beam, a pulse from a second laser beam is applied to the modified surrounding tissue region during the enhanced absorptivity time window of the modified surrounding tissue region to thus cauterize the modified surrounding tissue region. The pulse from the second laser beam is lower in intensity than the pulse from the first laser beam but longer in duration than the pulse from the first laser beam. At operation 206 pulses using both the first and second laser beams are controllably applied as needed to continue the simultaneous cutting of tissue and the cauterization of the surrounding modified tissue region. As explained above, if a single laser is used, the pulse type 106 shown in FIG. 4 may be used in which case both the cutting and the cauterizing may be accomplished with a single laser. It can be appreciated that the time durations of the first and second pulses are relatively short compared to laser beam stability from the handling of the operator. Thus, the pointing stability on the target region of the second laser pulse is almost ideal in all possible practical cases.

It will be appreciated that control over the intensity of the second laser pulses 18 or 106 needs to be closely maintained to be able to effectively achieve the desired cauterization. This intensity may need to be varied for different tissue types. The intensity of the second pulse may also be temporally modulated with parts of the pulse requiring higher intensities than other parts of the pulse during the duration of the pulse. In other words the pulse shape maybe more complex than the hat-top intensity profile shown in the FIG. 4.

Still further, it will be appreciated that the first and second laser beams described herein may be delivered using a fiber optic system. The above can be combined with other diagnostic instrumentation such as with spectroscopic characterization and/or imaging. Also, while the tissue has been described herein as human tissue, the systems and methods described herein are expected to have the same utility when applied to tissue from animals.

While various embodiments have been described, those skilled in the art will recognize modifications or variations which might be made without departing from the present disclosure. The examples illustrate the various embodiments and are not intended to limit the present disclosure. Therefore, the description and claims should be interpreted liberally with only such limitation as is necessary in view of the pertinent prior art.

What is claimed is:

1. A method for substantially cutting and cauterizing tissue during a surgical procedure, the method comprising:
using a first laser beam to irradiate a first area, the first laser beam being configured in intensity and duration to cut the tissue within the first area, the first laser beam further causing a temporary increase in an absorptivity of tissue in a second area which is in proximity to the first area; and
using a second laser beam to irradiate the second area while the temporary increase in absorptivity is occurring, the second laser beam having a different intensity than the first laser beam to cauterize tissue within the second area substantially simultaneously with the cutting of the tissue in the first area.

2. The method of claim 1, wherein:
using the first laser beam comprises using laser beam pulses each having a first duration to irradiate the first area; and
using the second laser beam comprises using laser beam pulses that each have a second duration, and wherein the second duration is longer than the first duration.

3. The method of claim 1, wherein using the second laser beam comprises using laser beam pulses which have an intensity which is less than an intensity of the first laser beam.

4. The method of claim 1, wherein using the first laser beam comprises using laser beam pulses each being less than 1 microsecond in duration.

5. The method of claim 4, wherein the first laser beam comprises laser beam pulses each having a duration of 10 picoseconds to 10 nanoseconds.

6. The method of claim 1, wherein using the second laser beam comprises using a laser beam having pulses between about 10 microseconds to about 100 microseconds in duration.

7. The method of claim 1, further comprising first using the first laser beam to generate a first series of pulses, and then using the second laser beam to generate a second series of pulses during the period of increased absorptivity induced by the pulses of the first beam.

8. The method of claim 1, further comprising using the first laser beam to generate a first series of pulses, and using the second laser beam to generate a second series of pulses that at least partially overlap with the first series of pulses, and which are each of a longer pulse duration than those of the first series of pulses.

9. The method of claim 1, wherein using the first and second laser beams comprises generating a series of first and second laser pulses using at least one of:
separate lasers to generate the first and second laser beams; or
a single laser to alternately generate the pulses of the first and second laser beams.

10. The method of claim 1, wherein an energy deposited into the second area by the second laser beam is proportional to the increased absorptivity of the tissue within the second area.

11. The method of claim 1, further comprising forming the first and second laser beams from a single series of laser pulses, where each said laser pulse includes:
a first portion forming a spike of a first intensity and a first duration; and
a second portion forming a tail having a second intensity less than the first intensity, and a second duration which is greater than the first duration, and wherein the second portion is generated within the time that the increased absorptivity of the tissue is occurring in the second area.

12. The method of claim 1, wherein an intensity of the second laser beam is temporally modulated such that portions of laser pulses of the second laser beam have differing intensities.

13. The method of claim 1, wherein the tissue is human tissue.

14. The method of claim 1, wherein the tissue is animal tissue.

15. A method for substantially cutting and cauterizing tissue during a surgical procedure, the method comprising:
using a first laser beam having a first series of laser pulses to irradiate a first area, the first laser pulses being configured in intensity and duration to cut the tissue within the first area, the first laser beam further causing a temporary increase in an absorptivity of tissue in a second area which is in proximity to the first area; and
using a second laser beam having a second series of laser pulses to irradiate the second area while the temporary increase in absorptivity is occurring, the second series of laser pulses each having a lower intensity than the pulses of the first series laser pulses and each being longer in duration than those of the first series of laser pulses, the second series of laser pulses cauterizing tissue within the second area substantially simultaneously with the cutting of the tissue in the first area.

16. The method of claim 15, wherein the first series of pulses and the second series of pulses overlap.

17. The method of claim 15, wherein an energy deposited into the second area by the second series of laser pulses is proportional to the increased absorptivity of the tissue within the second area.

18. The method of claim 15, wherein the first and second series of laser pulses are formed from a single train of laser pulses from a single laser device, with the first series of laser pulses representing a first portion of each pulse from the single train of laser pulses, and the second series of laser pulses representing a second portion of each one of the second series of laser pulses.

19. The method of claim 15, wherein an intensity of the second series of laser pulses is modulated to vary the intensity during each one of the pulses of the second series of laser pulses.

20. A system for substantially cutting and cauterizing tissue during a surgical procedure, the system comprising:
at least one laser configured to generate:
a first laser beam being configured in intensity and duration to cut tissue within a first area, the first laser beam further causing a temporary increase in an absorptivity of tissue in a second area which is in proximity to the first area; and
a second laser beam configured in intensity and duration to irradiate the second area while the temporary increase in absorptivity is occurring, the second laser beam having laser pulses which are of a different intensity and a different duration than laser pulses forming the first laser beam, to cauterize tissue within the second area substantially simultaneously with the cutting of the tissue in the first area.
